(19) United States
(12) Patent Application Publication
(10) Pub. No.: US 2025/0205503 A1
(43) Pub. Date: Jun. 26, 2025

(54) WIRELESS NEUROMODULATION SYSTEMS

(71) Applicant: BLUEWIND MEDICAL LTD., Herzlia (IL)

(72) Inventors: Yigal ELISHA, Holon (IL); Yaron Laufer, Zikhron Yaakov (IL); Michael Khmelev, Ramla (IL); Alex Schuster, Petah Tikva (IL); Gur Oron, Tel Aviv (IL)

(73) Assignee: BLUEWIND MEDICAL LTD., Herzlia (IL)

(21) Appl. No.: 18/973,265

(22) Filed: Dec. 9, 2024

Related U.S. Application Data
(60) Provisional application No. 63/612,594, filed on Dec. 20, 2023.

(51) Int. Cl. A61N 1/378; A61N 1/36

(52) U.S. Cl. CPC A61N 1/3787; A61N 1/36007

(57) ABSTRACT

An external control unit is provided that includes a power unit, which includes a power amplifier arranged to provide current to an antenna circuit; first and second parallel current paths; and a storage capacitor, connected to the first parallel current path and not to the second parallel current path. A microcontroller is configured to drive the power unit to charge the storage capacitor with the power from the battery at times other than during stimulation periods; during one or more non-stimulation periods, drive the power unit to provide the power from the battery to the power amplifier via the second parallel current path and not via the first parallel current path; and during the stimulation periods, drive the power unit to provide power from the storage capacitor to the power amplifier via the first parallel current path and not via the second parallel current path. Other embodiments are also described.

### CROSS-REFERENCE TO RELATED APPLICATIONS

The present application claims the benefit of US Provisional Application 63/612, 594, filed Dec. 20, 2023, which is assigned to the assignee of the present application and incorporated herein by reference.

### FIELD OF THE APPLICATION

Applications of the present invention relate to transmitting power and data to an implanted medical device.

### BACKGROUND OF THE APPLICATION

Electrical power can be transferred to a medical implant by magnetic induction. A current flowing through a coil produces a magnetic field, which, in turn, will induce a current in a second coil. A coil inside a medical implant can therefore act as a receiving coil, while a coil outside a patient's body can act as a transmitting coil. A current can be driven through the transmitting coil in order to induce an induced current in the receiving coil, thereby powering the medical implant.

U.S. Pat. No. 11,213,685 to Oron et al. which is incorporated herein by reference, describes apparatus for use with a medical implant having a receiving coil. A flexible housing to be placed against skin of a subject includes a flexible transmitting coil and control circuitry for driving a current through the transmitting coil to induce a current in the receiving coil. A sensor coupled to the circuitry determines divergence of a resonance frequency of the transmitting coil when flexed from a nominal resonance frequency of the transmitting coil, occurring in the absence of any forces applied to the transmitting coil. One or more electrical components coupled to the circuitry tune the resonance frequency of the transmitting coil. A switch is coupled to each of the electrical components, the switches including transistors having capacitances that depend on the voltage applied to each switch. The circuitry applies a voltage of 30-300 volts to each switch. Other applications are also described.

US Patent Application Publication 2023/0170138 to Oron et al., which is incorporated herein by reference, describes a housing placeable against skin of a subject that includes a transmitting coil. Battery-powered control circuitry including a power stage transmits power to an implant by activating the power stage to drive a current through the transmitting coil to induce an induced current in a receiving coil of the implant. A sensor indicates divergence of a real-time resonance frequency of the transmitting coil with respect to a reference resonance frequency of the transmitting coil at which (a) efficiency of the power stage is at least 70% of a maximum efficiency as a function of the resonance frequency, and (b) the current driven through the transmitting coil is less than 96% of a maximum current drivable through the transmitting coil as a function of the resonance frequency. The circuitry reduces the divergence by tuning the resonance frequency of the transmitting coil. Other applications are also described.

### SUMMARY OF THE APPLICATION

In accordance with some applications of the present invention, an electrostimulation system is provided that comprises an electrostimulator implant, which is implantable in a subject, and an external control unit (ECU), which is configured to be coupled to an external surface of the subject. By way of example and not limitation, the electrostimulator implant may comprise an implantable tibial nerve stimulator. The electrostimulator implant comprises a receiving coil, one or more electrodes, and implant circuitry. Typically, the electrostimulator implant does not comprise an internal power source and is instead powered and controlled by the ECU. The implant uses power received from the ECU to inject a current pulse into the surrounding tissue, for example to stimulate a nerve, e.g., the tibial nerve, such as for treating overactive bladder (OAB).

There is therefore provided, in accordance with an application of the present invention, an external control unit (ECU) for use with an electrostimulator implant that includes a receiving coil and is implantable in a subject, the ECU including:
(i) a housing;
(ii) a battery coupled to the housing;
(iii) an antenna circuit, including a transmitting coil;
(iv) a power unit, including:
   (a) a power amplifier, which is arranged to provide current to the antenna circuit;
   (b) first and second parallel current paths, which are arranged to convey power from the battery to the power amplifier; and
   (c) a storage capacitor, connected to the first parallel current path and not to the second parallel current path; and
(v) a microcontroller, which is configured to:
   transmit power to the electrostimulator implant in a plurality of stimulation pulse trains, by activating the power unit to provide the current to the transmitting coil of the antenna circuit to induce an induced current in the receiving coil, wherein each of the stimulation pulse trains includes a stimulation period and one or more non-stimulation periods,
   drive the power unit to charge the storage capacitor with the power from the battery at least at one or more times other than during the stimulation period of each of the stimulation pulse trains,
   during the one or more non-stimulation periods of each of the stimulation pulse trains, drive the power unit to provide the power from the battery to the power amplifier via the second parallel current path and not via the first parallel current path, and
   during the stimulation period of each of the stimulation pulse trains, drive the power unit to provide power from the storage capacitor to the power amplifier via the first parallel current path and not via the second parallel current path.

For some applications, the microcontroller is configured to drive the power unit to charge the storage capacitor with the power from the battery only at one or more times other than during the stimulation period of each of the stimulation pulse trains.

For some applications, the microcontroller is configured to set an amplitude of the stimulation period to be greater than each of one or more respective amplitudes of the one or more non-stimulation periods.

For some applications, the ECU is configured to transmit more energy during the stimulation period than during each of the one or more non-stimulation periods.

For some applications, the one or more non-stimulation periods include a control period before the stimulation period.

For some applications, the microcontroller is configured to encode data in pulses of the control period of the one or more non-stimulation periods.

For some applications, the one or more non-stimulation periods further include a discharge period after the stimulation period.

For some applications, the one or more non-stimulation periods include a discharge period after the stimulation period.

For some applications, the storage capacitor has a capacitance of at least 1 millifarad.

For some applications, the power amplifier includes a Class E power amplifier.

For some applications, the power unit is configured to provide higher power to the power amplifier during the stimulation period of each of the stimulation pulse trains than during the one or more non-stimulation periods of each of the stimulation pulse trains.

For some applications, the power unit is configured to provide, from the storage capacitor to the power amplifier during the stimulation period of each of the stimulation pulse trains, more power than the battery is capable of supplying.

For some applications, the power amplifier is configured to drive the antenna circuit at a single fixed frequency during the stimulation period and the non-stimulation periods of each of the stimulation pulse trains.

For some applications:
the antenna circuit further includes a compensation circuit in series with the transmitting coil, and the compensation circuit is configured to provide the antenna circuit with an adjustable resonance frequency, and
the microcontroller is configured to tune the antenna circuit, via the compensation circuit, to have a resonance frequency that does not match the fixed frequency at which the power amplifier drives the antenna circuit.

For some applications, the fixed frequency is 6.78 MHz.

For some applications:
the first parallel current path includes (a) a first first-path switch arranged between the battery and the storage capacitor, and (b) a second first-path switch arranged between the storage capacitor and the power amplifier, and
the microcontroller is configured to:
   drive the power unit to charge the storage capacitor with the power from the battery by closing the first first-path switch and opening the second first-path switch, and
   drive the power unit to provide power from the storage capacitor to the power amplifier via the first parallel current path by opening the first first-path switch and closing the second first-path switch.

For some applications, the power unit further includes a filter capacitor, which is arranged in the second parallel current path and not the first parallel current path.

For some applications, the filter capacitor has a capacitance of less than 5,000 nF.

For some applications, a capacitance of the storage capacitor is greater than a capacitance of the filter capacitor.

For some applications, a ratio of the capacitance of the storage capacitor to the capacitance of the filter capacitor is at least 1,000.

For some applications, the power unit further includes a DC/DC conversion module, which (a) includes one or two DC/DC converters, and (b) is arranged to receive the power from the battery and provide the power to the first and the second parallel current paths.

For some applications, the DC/DC conversion module includes exactly one DC/DC converter that is connected to both the first and the second parallel current paths.

For some applications, the microcontroller is configured to drive the power unit to charge the storage capacitor with the power from the battery via the DC/DC conversion module, at one or more times other than during the stimulation and non-stimulation periods of each of the stimulation pulse trains.

For some applications, the microcontroller is configured to set the DC/DC converter at a first output voltage when providing the power to the first parallel current path, and at a second output voltage when providing the power to the second parallel current path, the first output voltage higher than the second output voltage.

For some applications, the DC/DC conversion module includes:
a first DC/DC converter that is connected to the battery and the first parallel current path; and
a second DC/DC converter that is connected to the battery and the second parallel current path.

For some applications, the first and the second DC/DC converters have first and second output voltages, respectively, the first output voltage higher than the second output voltage.

For some applications, the first DC/DC converter includes a buck converter, and the second DC/DC converter includes a boost converter.

For some applications, the microcontroller is configured to drive the power unit to charge the storage capacitor with power from the battery via the first DC/DC converter at the one or more times other than during the stimulation period of each of the stimulation pulse trains, the one or more times including during at least one of the one or more non-stimulation periods of each of the stimulation pulse trains.

For some applications, an electrostimulation system is provided that includes the ECU and further includes the electrostimulator implant.

There is further provided, in accordance with an application of the present invention, an external control unit (ECU) for use with an electrostimulator implant that includes a receiving coil and is implantable in a subject, the ECU including:
(i) a housing;
(ii) a battery coupled to the housing;
(iii) an antenna circuit, including a transmitting coil;
(iv) a power unit, including a power amplifier, which is arranged to provide current to the antenna circuit; and
a microcontroller, which is configured to:
   transmit power to the electrostimulator implant in a plurality of stimulation pulse trains, by activating the power unit to provide the current to the transmitting coil of the antenna circuit to induce an induced current in the receiving coil, wherein each of the stimulation pulse trains includes (a) a stimulation period and (b) a non-stimulation data-transmission period, and
   encode data in a plurality of bursts during the non-stimulation data-transmission period, by:
      configuring each of the bursts to include an “on” period, during which power is transmitted at a frequency and an amplitude, and an “off” period, during which power is not transmitted, such that a burst duration of each of the bursts equals a sum of an “on” duration of the “on” period and an “off” duration of the “off” period of the burst, and
      transmitting single bits of the data in respective bursts, by representing each “0” value of the bits by a first burst duration, and each “1” value of the bits by a second burst duration, the second burst duration different from the first burst duration.

For some applications, the “off” duration of the bursts having the first burst duration equals the “off” duration of the bursts having the second burst duration.

For some applications, the “on” duration of the bursts having the first burst duration is different from the “on” duration of the bursts having the second burst duration.

For some applications, a ratio of the “off” duration to the burst duration of the bursts having the first burst duration is less than 0.5.

For some applications, a ratio of the “off” duration to the burst duration of the bursts having the second burst duration is less than 0.4.

For some applications, the “off” duration of each of the bursts is no more than 50 microseconds.

For some applications, the “on” period precedes the “off” period in each of the bursts.

For some applications, the “on” period follows the “off” period in each of the bursts.

For some applications, the non-stimulation data-transmission period is an entirely or a portion of a control period before the stimulation period of each of the stimulation pulse trains.

For some applications, the microcontroller is configured to set an amplitude of the stimulation period to be greater than an amplitude of the non-stimulation data-transmission period.

For some applications, the power unit is configured to provide higher power to the power amplifier during the stimulation period of each of the stimulation pulse trains than during the non-stimulation data-transmission period of each of the stimulation pulse trains.

For some applications, the power amplifier is configured to drive the antenna circuit at a single fixed frequency during the stimulation period and the “on” periods of the bursts of the non-stimulation data-transmission period of each of the stimulation pulse trains.

For some applications, an electrostimulation system is provided that includes the ECU and further includes the electrostimulator implant, which includes implant circuitry configured to decode the data encoded in the plurality of bursts by the microcontroller of the ECU.

For some applications:
the electrostimulator implant includes an energy storage element capable of storing sufficient energy to power operation of the electrostimulator implant for up to a maximum amount of time during the non-stimulation data-transmission period of each of the stimulation pulse trains, and
the “off” duration of each of the bursts is less than the maximum amount of time.

There is still further provided, in accordance with an application of the present invention, an electrostimulation system for application to a subject, the electrostimulation system including:
an external control unit (ECU), which includes (i) a housing; (ii) a battery coupled to the housing; (iii) an antenna circuit, including a transmitting coil; (iv) a power unit, including a power amplifier, which is arranged to provide current to the antenna circuit; and (v) a microcontroller; and
an electrostimulator implant, which is implantable in the subject, and which includes (i) a receiving coil; (ii) one or more electrodes; and (iii) implant circuitry,
wherein the microcontroller of the ECU is configured to:
   transmit energy to the electrostimulator implant in a plurality of pulse trains, by activating the power unit to provide the current to the transmitting coil of the antenna circuit to induce an induced current in the receiving coil, wherein each of the pulse trains includes (a) a lower-amplitude period followed by (b) a higher-amplitude period, and
   encode data in at least a portion of the lower-amplitude period of each of the pulse trains indicative of whether the higher-amplitude period is a stimulation period or a non-stimulation period, and
wherein the implant circuitry is configured to, during each of the pulse trains:
   receive and decode the encoded data,
   in response to the decoded data indicating that the higher-amplitude period is the stimulation period, drive the one or more electrodes to apply electrical stimulation to tissue of the subject using the energy received during the higher-amplitude period, and
   in response to the decoded data indicating that the higher-amplitude period is the non-stimulation period, not drive the one or more electrodes to apply electrical stimulation to tissue of the subject.

For some applications, the implant circuitry is configured to, in response to the decoded data indicating that the higher-amplitude period is the non-stimulation period, discharging at least a portion of the energy received during the higher-amplitude period without applying electrical stimulation to the tissue of the subject.

For some applications, during an ECU positioning period for positioning the transmitting coil of the ECU with respect to the receiving coil of the electrostimulator implant:
the microcontroller of the ECU is configured to encode the data indicative that the higher-amplitude period is the non-stimulation period, and
the implant circuitry is configured, in response to the decoded data indicating that the higher-amplitude period is the non-stimulation period, provide data, using at least a portion of the received energy, to the microcontroller of the ECU indicative of a position of the transmitting coil of the ECU with respect to the receiving coil of the electrostimulator implant.

For some applications, the microcontroller of the ECU is configured to configure a first portion of the pulse trains as stimulation pulse trains, and a second portion of the pulse trains as non-stimulations pulse trains interspersed among the stimulation pulse trains.

The present invention will be more fully understood from the following detailed description of embodiments thereof, taken together with the drawings, in which:

### BRIEF DESCRIPTION OF THE DRAWINGS

FIG. 1 is a schematic illustration of an electrostimulation system, in accordance with an application of the present invention;

FIGS. 2A and 2B are schematic block diagrams of elements of the electrostimulation system of FIG. 1, in accordance with respective applications of the present invention;

FIG. 3 is a schematic illustration of an implant start-up protocol, in accordance with an application of the present invention;

FIG. 4 is a schematic illustration of a plurality of stimulation pulse trains, in accordance with an application of the present invention;

FIG. 5 is a schematic illustration of a non-stimulation data-transmission period of a pulse train, in accordance with an application of the present invention; and

FIG. 6 is a schematic illustration of an implementation of dummy stimulation pulse trains, in accordance with an application of the present invention.

### DETAILED DESCRIPTION OF APPLICATIONS

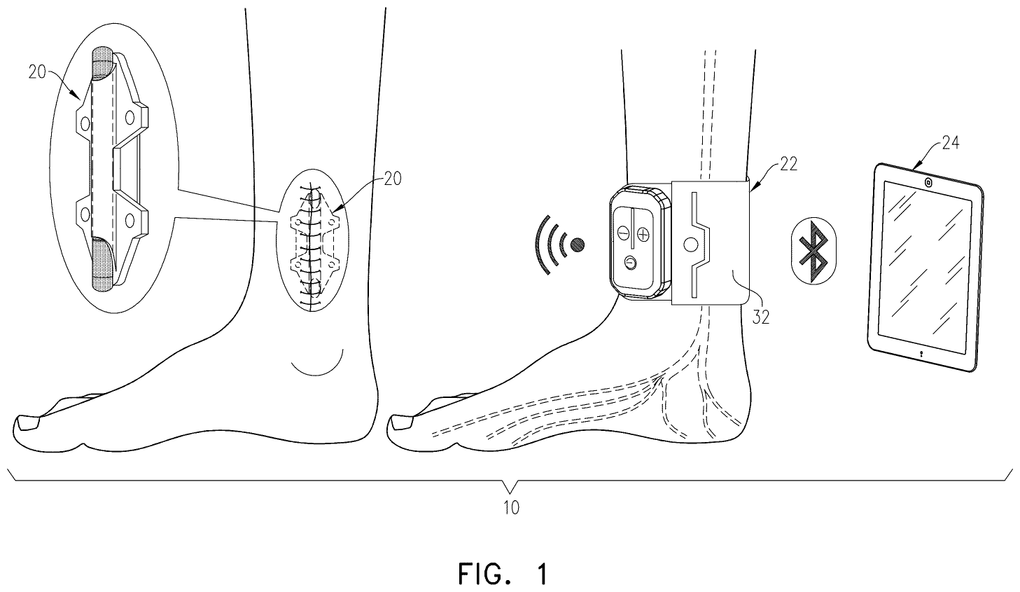

FIG. 1 is a schematic illustration of an electrostimulation system 10, in accordance with an application of the present invention. Electrostimulation system 10 comprises an electrostimulator implant 20, which is implantable in a subject, and an external control unit (ECU) 22, which is configured to be coupled to an external surface of the subject. By way of example and not limitation, electrostimulator implant 20 may comprise an implantable tibial nerve stimulator, which optionally implements any of the techniques described in the patents and patent application publications incorporated by reference hereinbelow. For example, the implantable tibial nerve stimulator may be implanted in a lower leg of a subject in the vicinity of the tibial neurovascular bundle, and ECU 22 may be wrapped around the leg at the implant site.

Optionally, electrostimulation system 10 further comprises a clinician programmer 24, which is configured to allow a clinician to set and adjust treatment parameters, such as current amplitude, current pulse width, polarity, repetition rate (frequency) of the neuro-stimulation pulses, treatment session duration, and maximum daily treatment duration, and to transmit the parameters for storage in ECU 22. Typically, ECU 22 automatically stops treatment at the end of the set treatment duration and/or upon meeting the maximum daily treatment duration. For example, the duration of a treatment session may be about half an hour and may be performed twice per day, and the total daily treatment duration may be two hours. For example, ECU 22 may be programmed to apply nerve stimulation using stimulation pulse trains 52 (as described hereinbelow) at a pulse-train frequency of 2-30 Hz, i.e., 2-30 stimulation pulse trains per second.

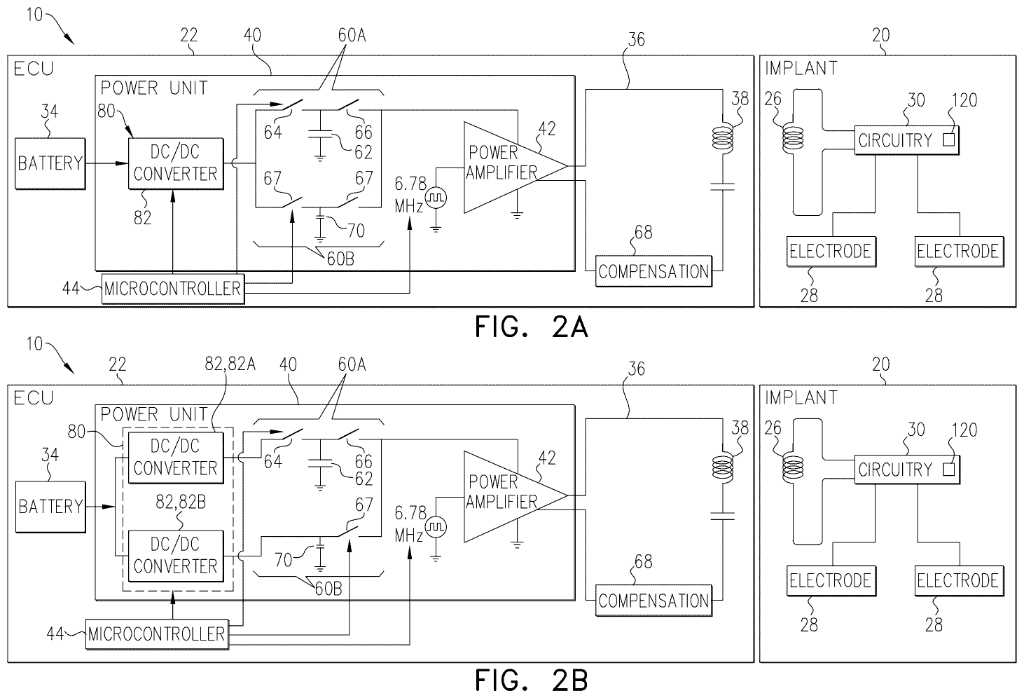

Reference is still made to FIG. 1, and is further made to FIGS. 2A and 2B, which are schematic block diagrams of elements of electrostimulation system 10, in accordance with respective applications of the present invention.

Electrostimulator implant 20 comprises a receiving coil 26, one or more electrodes 28, and implant circuitry 30. Typically, electrostimulator implant 20 does not comprise an internal power source and is instead powered by ECU 22. Electrostimulator implant 20 uses power received from ECU 22 to inject a current pulse into the surrounding tissue, for example to stimulate a nerve, e.g., the tibial nerve, such as for treating overactive bladder (OAB).

The communication between ECU 22 and electrostimulator implant 20 enables ECU 22 to control and monitor electrostimulator implant 20. Since the implant is small and limited in resources, much of the control and monitoring may be performed at ECU 22 side using the communication channel. For example, ECU 22 may send the relevant treatment parameters to the implant or instruct the implant to apply certain types of pulses with different functionalities. During operation, the implant may measure its internal signals and communicate them back to ECU 22, so that the functionality of electrostimulation system 10 can be properly monitored, and safe operation can be ensured.

ECU 22 typically comprises:
a housing 32 (shown and labeled in FIG. 1), which is optionally wearable by the subject;
a battery 34, which is coupled to housing 32 (either within housing 32 or external to housing 32); typically, battery 34 is rechargeable;
an antenna circuit 36, which comprises a transmitting coil 38;
a power unit 40, which comprises a power amplifier 42, which is arranged to provide current to antenna circuit 36; and.
a microcontroller 44 (the circuitry and/or functionality of microcontroller 44 may be provided in a single microcontroller, or distributed among several interconnected microcontrollers).

Typically, ECU 22 further comprises additional electronic components, including, by way of example and not limitation, one or more filters and one or more capacitors, as known in the wireless transmitter art.

Microcontroller 44 is configured to transmit power to electrostimulator implant 20 in a plurality of pulse trains, by activating power unit 40 to provide the current to transmitting coil 38 of antenna circuit 36 to induce an induced current in receiving coil 26 by inductive resonant coupling, e.g., at 6.78 MHz. Receiving coil 26 creates oscillating voltage due to the induced time varying magnetic flux, which is then rectified and filtered to provide a DC voltage for the operation of the implant. Variations in the inductance of transmitting coil 38 cause variations in the resonance frequency of antenna circuit 36, thereby causing the antenna circuit to act as a variable load for power amplifier 42. Changes in inductance of the transmitting coil may occur, for example, due to the transmitting coil flexing, or due to the transmitting coil being in close proximity to metal in the environment of the patient.

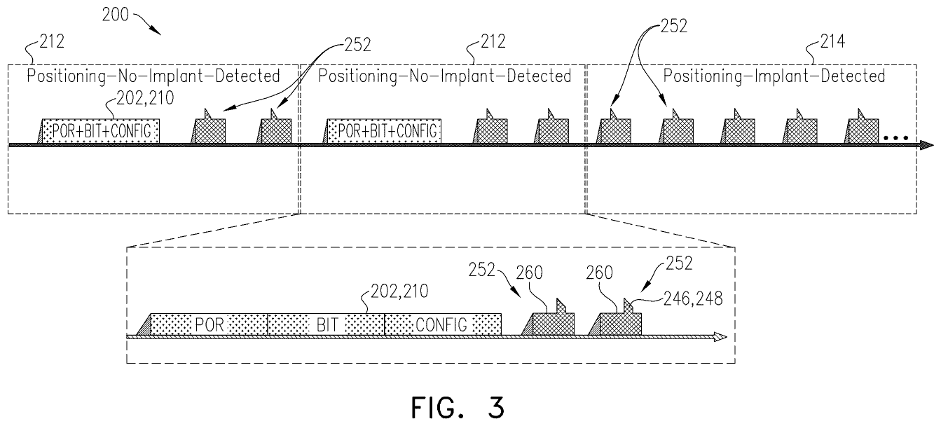

ECU 22 is used to perform treatment sessions. To start a treatment session, the patient may wrap ECU 22 around the leg using a leg band and presses an on button. In response, ECU 22 starts to transmit the pulses to locate the implant, initiate it, and provide power for its operation, such as described hereinbelow with reference to FIG. 3.

### Types of Pulse Trains

Reference is made to FIG. 3, which is a schematic illustration of an implant start-up protocol 200, in accordance with an application of the present invention.

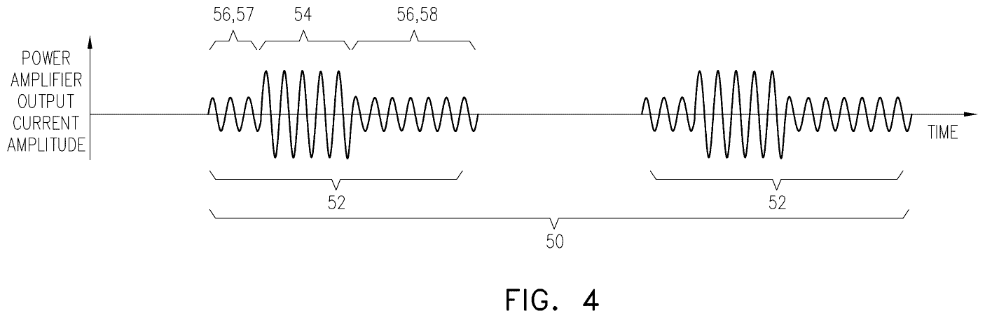

Reference is also made to FIG. 4, which is a schematic illustration of a plurality 50 of stimulation pulse trains 52 of the current generated by power unit 40, in accordance with an application of the present invention.

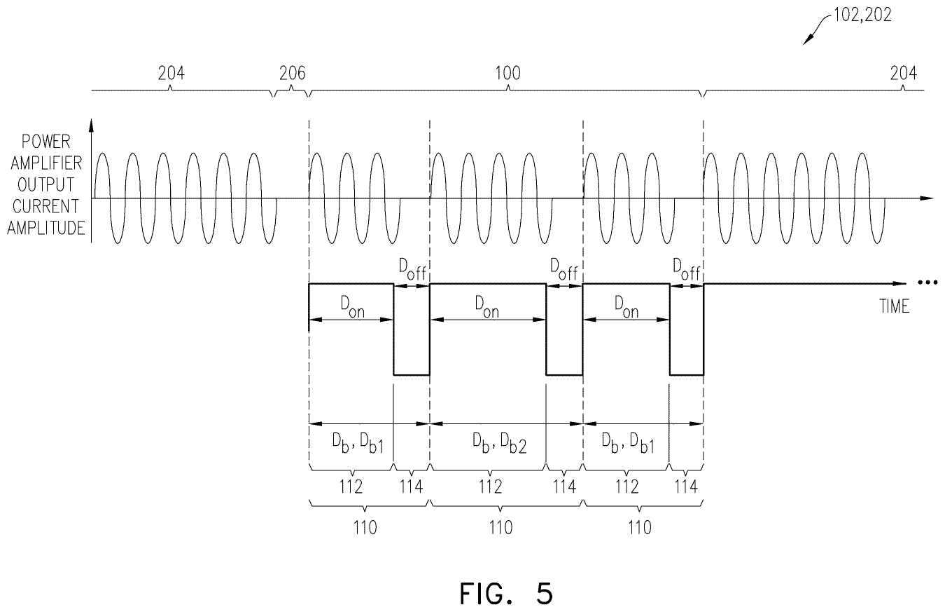

Microcontroller 44 of ECU 22 is typically configured to drive transmitting coil 38 of antenna circuit 36 to generate different types of pulse trains for different purposes, as described in detail hereinbelow. The types of pulse trains typically include, but are not limited to:
data pulse trains 202, in which ECU 22 transmits data to implant 20, such as described hereinbelow with reference to FIG. 3; data pulse trains 202 typically have only a single amplitude throughout the pulse train (with the optional exception of “off” periods for data encoding, such as described hereinbelow with reference to FIG. 5), and typically transmit only enough power for operations of implant circuitry 30, but not enough power for the implant to apply tissue stimulation; and
stimulation pulse trains 52, which typically include enough power for electrostimulator implant 20 to apply tissue stimulation, as well as a small amount of data, such as described hereinbelow with reference to FIG. 4; stimulation pulse trains 52 may contain more than one amplitude, such as one or more lower-amplitude periods in which data is transferred with minimal power, and one or more higher-amplitude periods (e.g., exactly one higher-amplitude period), in which power is transmitted (typically without data).

### Data Pulse Trains

Microcontroller 44 of ECU 22 is typically configured to drive transmitting coil 38 of antenna circuit 36 to generate different types of data pulse trains 202 for different purposes, as described in detail hereinbelow. The types of data pulse trains 202 typically include:
initialization pulse trains 210, such as described hereinbelow with reference to FIG. 3, and
configuration pulse trains, such as described hereinbelow.

### Treatment Sessions

Typically, each treatment session includes one or more of the following stages: a compensation adjustment stage, a positioning stage, a ramp-up stage, and a treatment stimulation stage. Optionally, treatment sessions do not necessarily include all of these stages.

### Compensation Adjustment Stage

For some applications, ECU 22 is configured to perform compensation adjustment after ECU 22 has been positioned for stimulation, such as wrapped around the patient's leg. The compensation adjustment may improve power transfer efficiency. For some applications, ECU 22 performs the compensation adjustment by setting an initial value for compensation circuit 68, described hereinbelow with reference to FIGS. 2A-B. The compensation adjustment may, for example, be performed using techniques described in one or more of the patents and/or patent applications incorporated hereinbelow by reference. Optionally, ECU 22 repeats the compensation adjustment stage upon the conclusion of the positioning stage, such as to compensate for movement of the antenna during the repositioning stage.

### Positioning Stage

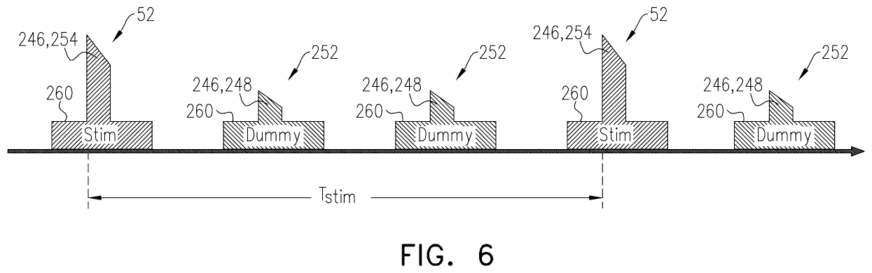

Reference is again made to FIG. 3. After the above-described optional compensation adjustment stage, each treatment session continues with a positioning stage. The positioning stage begins with a start-up phase 212 (labeled “Positioning-No-Implant-Detected” in FIG. 3), prior to detection of implant 20 by ECU 22. During start-up phase 212 ECU 22 delivers power to boot implant 20. ECU 22 is configured to transmit initialization pulse trains 210 during implant start-up protocol 200, as well as on implant reset (such as if the implant loses power during a treatment session), and, optionally, during implant built-in testing. Optionally, ECU 22 sends dummy stimulation pulse trains 252 interspersed with initialization pulse trains 210, in order to provide power to the implant, such as described hereinbelow with reference to FIGS. 3 and 6. ECU 22 sends initialization pulse trains 210 until ECU 22 receives a signal encoding data feedback from implant 20, indicating that ECU 22 has achieved wireless communication with implant 20, and that implant 20 has been detected by ECU 22. By way of illustrative example, FIG. 3 shows exactly two initialization pulse trains 210, although in practice ECU 22 often sends more such trains.

Initialization pulse trains 210 (for example, in the configuration period described hereinbelow) encode data that are used for implant initialization and/or setting of relevant stimulation parameters of the implant. ECU 2 sends bits representing implant identification information and stimulation parameters to the implant. The implant response (e.g., via load modulation) includes bits that inform ECU 22 that values sent were correctly received and data of measurements performed by the implant on its electronics.

For example, each of initialization pulse trains 210 may include the following:
a Power On Reset (POR) period—power to wake up and reset implant circuitry 30; typically, the POR period does not include any data,.
a Built In Test (BIT) period—power provided for implant 20 to upload implant software from nonvolatile memory to program microcontroller RAM, and, optionally, start a built-in test in which the implant runs its internal tests to confirm functionality; typically, the BIT period does not include any data, and/or
a configuration (CONFIG) period—power provided while implant 20 exchanges information with ECU 22 (addressed by serial number of the implant).

After ECU 22 detects implant 20, ECU 22 transitions to a position-optimizing phase 214 of the positioning stage (labeled “Positioning-Implant-Detected” in FIG. 3). During positioning-optimizing phase 214, the patient optimizes the positioning of ECU 22 with respect to the implant and confirms this placement. During positioning-optimizing phase 214, ECU 22 transmits only dummy stimulation pulse trains 252 to power implant 20 (as described hereinbelow with reference to FIGS. 3 and 6), and receives data back from the implant during these pulse trains to enable adjusting of the position of the ECU by the patient. For example, the data may be indicative of the DC voltage obtained in implant 20 by the wireless power received, as an indicator of the positioning quality. This data may be presented to the patient as a measure of position quality, such as audially and/or visually, and the patient improves the position of the ECU until adequate positioning is obtained.

Typically, if at any time during a treatment session communication between ECU 22 and implant 20 is lost for more than a certain duration, ECU 22 returns to the positioning stage, in which the ECU indicates to the subject that the relative position between ECU 22 and implant 20 must be improved to provide better power transfer (and communication).

### Ramp-up Stage

Once proper positioning of ECU 22 is performed by the patient, ECU 22 begins the ramp-up stage. The ramp-up stage is used to prevent a sudden jump in patient sensation from no sensation during the positioning stage to the prescribed stimulation level sensation during the treatment stimulation stage. During the ramp-up stage, neurostimulation is slowly increased from low stimulation current amplitude to the prescribed amplitude. The gradual increase of the amplitude is performed in steps to allow the patient to adapt to the final treatment level.

For some applications, during the ramp-up stage, ECU 22 transmits configuration pulse trains, which include data indicative of the change in parameter(s) (e.g., amplitude) at each step of the ramp-up stage. For example, the configuration pulse trains may include only the configuration (CONFIG) period of the above-described initialization pulse trains 210. After transmitting each configuration pulse train, ECU 22 transmits stimulation pulse trains to provide power to implant 20 for applying tissue stimulation at the set level for the duration of the ramp step. The ECU then transmits another configuration pulse train to set the next level of the ramped stimulation, and this process is repeated until the ramp-up reaches the treatment amplitude level set by the clinician for the treatment. Optionally, a few configuration pulse trains are applied to check that the final level is as set, and ECU 22 begins the treatment stimulation stage.

### Treatment Stimulation Stage

The treatment stimulation stage starts at the end of the ramp-up stage and includes stimulation pulse trains 52 having the set stimulation amplitude (and any other set treatment parameters) that were set at the end of the ramp-up stage. The stimulation pulse trains are provided at the required repetition rate, as programmed by the clinician. For example, the duration of the stimulation stage may be thirty minutes (which also includes the ramp-up stage). At the end of this period, ECU 22 typically operates for up to one second to enable the removal (discharge) of any remnant tissue charge.

(If the time between consecutive pulse trains is so long that implant 20 does not have sufficient power to remain in sleep mode, the implant will require another complete start-up stage, as described above. In order to avoid this repetition of the start-up stage, dummy stimulation pulse trains 252 may be inserted in between stimulation pulse trains 52 to provide power (without stimulation), in order to shorten the duration of the sleep mode, such as described hereinbelow with reference to FIGS. 3 and 6.)

For some applications, ECU 22 is configured to allow the patient to adjust one or more of the stimulation parameters (e.g., the amplitude) of the tissue stimulation during the treatment stimulation stage. For example, the clinician may preconfigure a limited set of values of the parameters from the which the patient may choose, such as by pressing increase and decrease stimulation user control buttons. For some of these applications, ECU 22 transmits the new stimulation parameter(s) using a configuration pulse train, which may include only the configuration (CONFIG) period (without the remainder of an initialization pulse train 210).

Reference is again made to FIG. 4. Each of stimulation pulse trains 52 includes stimulation period 54 and one or more non-stimulation periods 56. By way of example, two stimulation pulse trains 52 are illustrated in FIG. 4, although during operation typically thousands stimulation pulse trains 52 are transmitted by ECU 22 during each treatment session (e.g., 2,000-100, 000 pulses during a 30-minute treatment session). ECU 22 is configured to transmit stimulation pulse trains 52 (as well as the other types of pulse trains described hereinabove) via antenna circuit 36.

Because ECU 22 is battery-operated, its available energy and power are limited. Therefore, the wireless power transmission scheme is adapted to minimize power consumption. For some applications, ECU 22 delivers the power to implant 20 by transmitting stimulation pulse trains 52 as very brief RF pulse trains. The repetition rate of the pulse trains is typically adapted to the required nerve stimulation frequency (typically, up to 30 Hz).

For some applications, the one or more non-stimulation periods 56 include a control period 57 before stimulation period 54 and/or a discharge period 58 after stimulation period 54. For applications in which the one or more non-stimulation periods 56 include control period 57, ECU 22 typically transmits power to wake up implant circuitry 30 during control period 57 of each stimulation pulse train 52. In addition, ECU 22 typically sends data to implant 20 during control period 57 of each stimulation pulse train 52, for example using the techniques described hereinbelow. This data transfer may enable identification by the implant of the power source as part of ECU 22 (such as to avoid inadvertent operation of the implant using “environmental” 6.78 MHz radiation). For example, ECU 22 may transmit an identifier code that is checked by implant 20 against the identifier code provided during start-up for the implant, in order for the implant to confirm that it is being powered by the ECU and not stray environmental radiation.

During stimulation period 54, ECU 22 is typically configured to transmit higher power, in order to provide implant 20 with sufficient energy for the injection of the neurostimulation current pulse into the tissue. Implant 20 uses the power received during stimulation period 54 to apply tissue stimulation. For example, implant 20 may be configured to apply the tissue stimulation in one or more biphasic pulses (e.g., exactly one biphasic pulse) per stimulation period 54. For example, each biphasic pulse may include a generally rectangular constant-current-amplitude stimulation portion during the stimulation period 54, followed by a discharge ramp-down period of opposite polarity. The discharge ramp-down period may occur during stimulation period 54, during discharge period 58, or partially during stimulation period 54 and partially during discharge period 58. For example, the pulse width of the generally rectangular constant-current-amplitude portion of each biphasic pulse applied by the implant may be 190-790 microseconds, and the constant-current amplitude of the generally rectangular constant-current-amplitude portion of each biphasic pulse applied by the implant may be up to 10 milliamps.

After application of the tissue stimulation, implant 20 typically responds by communicating to ECU 22, during discharge period 58, bits of data of measurements performed on the implant's internal electronics, such as during stimulation period 54 and/or during a portion of discharge period 58 before the implant transmits the feedback to the ECU. These implant measurement data are analyzed by ECU 22 to determine whether the implant is properly functioning.

This multi-level pulse train shape may allow ECU 22 to provide the amount of the power required at each stage, and not use the maximum amount of power throughout the entire pulse train duration, thus minimizing power and energy consumption. This multi-level pulse train shape may also lower heat dissipation within implant 20 and ECU 22, reduce the exposure of the human tissue to the electromagnetic field and the associated specific absorption rate (SAR), and/or lower the electromagnetic emissions from ECU 22.

For applications in which the one or more non-stimulation periods 56 include discharge period 58, during discharge period 58, ECU 22 transmits power at a lower level than during stimulation period 54 (this lower level may be the same as or different from the level used during control period 57), in order to enable implant 20 to discharge the tissue and communicate with ECU 22.

In addition, implant 20 may send data to ECU 22 during discharge period 58, which may enable ECU 22 to closely monitor the implant operation. For example, because implant 20 typically does not comprise an internal power source, the communication from the implant to ECU 22 may be performed by load modulation, as known in the wireless data transmission art, during discharge period 58.

Transmitting coil 38 of ECU 22 and receiving coil 26 of implant 20 are inductively coupled, such that a change of the implant load affects the RF current at transmitting coil 38 of ECU 22 while ECU 22 drives the transmitting coil with a constant voltage. For some applications, ECU 22 comprises an additional small antenna, coupled to the transmitting antenna, which receives the RF signal from the implant, and ECU 22 demodulates and decodes sensed variations in the analog signal (caused by the implant load modulation) into digital data according to an encoding scheme.

Typically, the duration of stimulation period 54 is shorter than the duration of control period 57 and shorter than the duration of discharge period 58. For example, the duration of control period 57 may be about 3 milliseconds, of which about 1 millisecond is used for data transfer and the remainder is used for power transfer; the duration of stimulation period 54 may vary according to the set current pulse width (e.g., 0.3-1 milliseconds); and/or the duration of discharge period 58 may be 9-10 milliseconds, for a total duration of each stimulation pulse trains 52 of about 10-20 milliseconds, e.g., about 15 milliseconds.

Since the clocks of ECU 22 and implant 20 are not synchronized, ECU 22 typically identifies the beginning and the end of feedback from implant 20. For example, this may be achieved by implant 20 sending a fixed string of bits as prefix and suffix of the feedback. ECU 22 samples the RF current envelope in a certain time slot during discharge period 58, identifies the prefix and suffix, and divides the time between them to the number of bits expected (for each type of data transmission). ECU 22 then assigns a bit value to each of the bits based on the sampled envelope during each bit period. Finally, ECU 22 calculates a checksum from the bits and compared to the checksum that is delivered as a part of the data transmission to confirm that the conversion is properly performed.

Typically, the power level during each period and its specific duration is set based on the period's requirements. Typically, during control period 57 the power level is lower, sufficient to wake up and keep implant circuitry 30 powered. During stimulation period 54 the power level is higher so the current injection may be supported. The duration of stimulation period 54 is determined by the width of the required neurostimulation current pulse (typically with additional margins). Finally, the power level during discharge period 58 is typically lower, sufficient to keep the critical electronics power up, to enable feedback communication from implant 20 to ECU 22, and for implant 20 to set itself into a low power consumption sleep mode in which it remains until the next transmitted pulse arrives.

Reference is now made to FIGS. 1-4. In an application of present invention, power unit 40 further comprises (shown in FIGS. 2A-B):
first and second parallel current paths 60A and 60B, which are arranged to convey power from battery 34 to power amplifier 42; and.
a storage capacitor 62, connected to first parallel current path 60A and not to second parallel current path 60B.

Battery 34 can supply only a certain maximum level of power. For example, battery 34 may be able to supply the lower power level required for the one or more non-stimulation periods 56 (e.g., control period 57 and/or discharge period 58) of a stimulation pulse train 52, but may be unable to directly provide enough power for stimulation period 54 of the stimulation pulse train 52. In this application of the present invention, in order to overcome this limitation of battery 34, energy is stored in storage capacitor 62 (typically at a slow rate) and then used to drive power amplifier 42 during stimulation period 54. To this end, microcontroller 44 is configured to:
drive power unit 40 to charge storage capacitor 62 with the power from battery 34 at least at one or more times other than during stimulation period 54 of each of stimulation pulse trains 52,.
during the one or more non-stimulation periods 56 of each of stimulation pulse trains 52 (e.g., control period 57 and/or discharge period 58), drive power unit 40 to provide the power from battery 34 to power amplifier 42 via second parallel current path 60B and not via first parallel current path 60A (i.e., without first storing the power in storage capacitor 62), and
during stimulation period 54 of each of the stimulation pulse trains 52, drive power unit 40 to provide power from storage capacitor 62 to power amplifier 42 via first parallel current path 60A (i.e., the portion of first parallel current path 60A to the right of storage capacitor 62 in FIGS. 2A-B) and not via second parallel current path 60B.

Thus, during the one or more non-stimulation periods 56 of each of stimulation pulse trains 52 (e.g., control period 57 and/or discharge period 58), power is provided from battery 34 to power amplifier 42 without first storing energy in storage capacitor 62. By contrast, providing power from storage capacitor 62 during both stimulation period 54 and the one or more non-stimulation periods 56 would require a larger storage capacitor than in the implementation described herein.

Typically, microcontroller 44 is configured to drive power unit 40 to charge storage capacitor 62 with the power from battery 34 only at one or more times other than during stimulation period 54 of each of stimulation pulse trains 52, such as only at one or more times other than during stimulation pulse trains 52.

Alternatively, microcontroller 44 is configured to drive power unit 40 to charge storage capacitor 62 with the power from battery 34 at the same time that storage capacitor 62 is discharging to provide power to power amplifier 42.

Typically, ECU 22 is configured to set an amplitude of stimulation period 54 to be greater than each of one or more respective amplitudes of the one or more non-stimulation periods 56. Alternatively or additionally, for some applications, ECU 22 is configured to transmit more energy during stimulation period 54 than during each of the one or more non-stimulation periods 56.

For some applications, the one or more non-stimulation periods 56 include control period 57 before stimulation period 54. For some of these applications, microcontroller 44 is configured to encode data in pulses of control period 57 of the one or more non-stimulation periods 56, such as by turning on and off the oscillator input that provides the fixed frequency signal to power amplifier 42. For example, the data may be encoded using techniques described hereinbelow with reference to FIG. 5, or using other techniques that are known in the art.

Alternatively or additionally, for some applications, the one or more non-stimulation periods 56 further include discharge period 58 after stimulation period 54.

The two parallel current paths 60A and 60B allow ECU 22 to quickly switch between two different voltages (low and high) within a single stimulation pulse train 52.

For some applications, ECU 22 is configured to transmit data pulse trains 202 (such as the above-described initialization pulse trains 210 and/or configuration pulse trains) by driving power unit 40 to provide the power from battery 34 to power amplifier 42 via second parallel current path 60B and not via first parallel current path 60A (i.e., without first storing the power in storage capacitor 62).

For some applications, storage capacitor 62 has a capacitance of at least 1 millifarad, e.g., at least 2 millifarad, such as at least 3 millifarad. Typically, the capacitance is selected based on the required DC current of power amplifier 42 during the stimulation and the voltage drop that is considered reasonable during the stimulation period.

For some applications, power amplifier 42 comprises a Class E power amplifier. This type of power amplifier is characterized by inherently high efficiency (compared to linear power amplifiers of Classes A, B and C) and the ability to control the output frequency, which is required to meet regulatory requirements regarding the emission band. The output level of power amplifier 42 (i.e., the amplitude of the RF current) can be adjusted by changing the DC voltage supplied to the amplifier, such as by DC/DC conversion module 80, described hereinbelow.

For some applications, power unit 40 is configured to provide higher power to power amplifier 42 during stimulation period 54 of each of stimulation pulse trains 52 than during the one or more non-stimulation periods 56 of each of stimulation pulse trains 52. Alternatively or additionally, for some applications, power unit 40 is configured to provide, from storage capacitor 62 to power amplifier 42 during stimulation period 54 of each of stimulation pulse trains 52, more power than battery 34 is capable of supplying.

For some applications, power amplifier 42 is configured to drive antenna circuit 36 at a single fixed carrier wave frequency during stimulation period 54 and the non-stimulation periods 56 of each of stimulation pulse trains 52. Thus, the carrier wave frequency is the same in stimulation periods 54 as in non-stimulation periods 56 schematically illustrated in FIG. 4, and is the same in the “on” periods 112 as in the one or more non-data power-only portions 204 of pulse train 102 schematically illustrated in FIG. 5. (For example, the single fixed carrier wave frequency may be 6.78 MHz, as described hereinabove and hereinbelow.)

For some applications, first parallel current path 60A comprises (a) a first first-path switch 64 arranged between battery 34 and storage capacitor 62, and (b) a second first-path switch 66 arranged between storage capacitor 62 and power amplifier 42. Microcontroller 44 is configured to:
drive power unit 40 to charge storage capacitor 62 with the power from battery 34 by closing first first-path switch 64 and opening second first-path switch 66, and.
drive power unit 40 to provide power from storage capacitor 62 to power amplifier 42 via first parallel current path 60A by opening first first-path switch 64 and closing the second first-path switch 66.

Alternatively or additionally, for some applications, second parallel current path 60B comprises one or more second-path switches 67 (e.g., two second-path switches 67, as shown in FIG. 2A, or exactly one second-path switch 67, as shown in FIG. 2B) arranged between battery 34 and power amplifier 42. Microcontroller 44 is configured to close the one or more second-path switches 67 when driving power unit 40 to provide the power from battery 34 to power amplifier 42 via second parallel current path 60B, and to open the one or more second-path switches 67 when not driving power unit 40 to provide the power from battery 34 to power amplifier 42 via second parallel current path 60B. Thus, during stimulation period 54, DC/DC conversion module 80 (and battery 34) are isolated from power amplifier 42. For some applications, power unit 40 further comprises a filter capacitor 70, which is arranged in second parallel current path 60B and not first parallel current path 60A. For example:
filter capacitor 70 may have a capacitance of less than 5, 000 nF, such as less than 3, 000 nF, and/or.
a capacitance of storage capacitor 62 may be greater than a capacitance of filter capacitor 70; e.g., a ratio of the capacitance of storage capacitor 62 to the capacitance of filter capacitor 70 may be at least 1,000.

For some applications, power unit 40 further comprises a DC/DC conversion module 80, which comprises one or two DC/DC converters 82. DC/DC conversion module 80 is arranged to receive the power from battery 34 and provide the power to first and second parallel current paths 60A and 60B, adjusting the voltage provided by battery 34 to the voltage required by power amplifier 42.

For some of these applications, such as shown in FIG. 2A, DC/DC conversion module 80 comprises exactly one DC/DC converter 82 that is connected to both first and second parallel current paths 60A and 60B. For example, DC/DC converter 82 may comprise a buck-boost DC/DC converter.

For some of these applications, microcontroller 44 is configured to drive power unit 40 to charge storage capacitor 62 with the power from battery 34 via DC/DC conversion module 80, at one or more times other than during the stimulation periods 54 and non-stimulation periods 56 of each of stimulation pulse trains 52. In these applications, microcontroller 44 is typically configured to set the DC/DC converter 82 at a first output voltage when providing the power to first parallel current path 60A, and at a second output voltage when providing the power to second parallel current path 60B, the first output voltage higher than the second output voltage. For example, DC/DC converter 82 may comprise a buck/boost converter.

For others of these applications, such as shown in FIG. 2B, DC/DC conversion module 80 comprises:
a first DC/DC converter 82, 82A that is connected to battery 34 and first parallel current path 60A; and
a second DC/DC converter 82, 82B that is connected to battery 34 and second parallel current path 60B.

For some of these applications, first and second DC/DC converters 82A and 82B have first and second fixed output voltages, respectively, the first output voltage higher than the second output voltage. For of some these applications, first DC/DC converter 82A comprises a boost converter, and second DC/DC converter 82B comprises a buck converter.

For others of these applications, one or both DC/DC converters 82A and 82B are configured to provide an adjustable output voltage, e.g., comprise respective buck/boost converters. For example, first DC/DC converter 82A may provide an adjustable output voltage (e.g., comprise a buck/boost converter), and second DC/DC converter 82B may provide a fixed output voltage.

For some of these applications, microcontroller 44 is configured to drive power unit 40 to charge storage capacitor 62 with power from battery 34 via first DC/DC converter 82A at the one or more times other than during stimulation period 54 of each of stimulation pulse trains 52, the one or more times including during at least one of the one or more non-stimulation periods 56 of each of stimulation pulse trains 52.

For some applications, the initial charging of storage capacitor 62 (at the start of operation) is performed as follows. Standard DC/DC converters are designed to operate on a resistive load, and to provide a voltage and current set by the ratio of the voltage to resistance. However, at the beginning of operation storage capacitor 62 is completely depleted and has no charge, so its voltage is zero. Trying to operate a DC/DC converter at a constant voltage will result in an “infinite” current spike, since there is no limiting current element such as a resistor. Including a resistor, while solving this problem, will cause constant power dissipation and additional power consumption and thus is preferably avoided. Instead, in some applications of the present invention, microcontroller 44 is configured to drive the charging of DC/DC converter 82 in a current controlled mode. The voltage is slowly increased from zero to the target voltage, while monitoring the charging current and keeping it constrained below the current limit of the converter. The current may be kept constant by setting a fixed voltage increase rate I=C dV/dt.

For some applications in which DC/DC conversion module 80 comprises first and second DC/DC converters 82A and 82B, such as shown in FIG. 2B, first DC/DC converter 82A comprises a boost converter, such as described above (and second DC/DC converter 82B comprises a buck converter). In these application, first DC/DC boost converter 82A cannot provide a voltage less than the battery voltage (because boost converters increase the voltage), and thus its direct connection to uncharged storage capacitor 62 will not result in the slow rise in voltage that may help prevent a current spike. First DC/DC boost converter 82A thus cannot provide the initial low voltage necessary to slowly charge storage capacitor 62 during its initial charging. Thus, for some of these applications, storage capacitor 62 is:
slowly charged up to the battery voltage using second DC/DC buck converter 82B, such as by closing second-path switch 67 and second first-path switch 66, and opening first first-path switch 64, and.
subsequently, slowly charged from the battery voltage to the target voltage using first DC/DC boost converter 82A, such as by opening second-path switch 67 and second first-path switch 66, and closing first first-path switch 64.

To keep power amplifier 42 non-operating while second-path switch 67 and second first-path switch 66 are closed during the first phase of slow charging described above, power unit 40 may comprise another switch along the DC voltage path to power amplifier 42, or the oscillator input may be disabled so that no RF current oscillation is produced.

For other applications in which DC/DC conversion module 80 comprises first and second DC/DC converters 82A and 82B and first DC/DC converter 82A comprises a buck/boost converter, such as described above, DC/DC converter 82A may be used for the entire slow charging of storage capacitor 62 by initially adjusting the output voltage of DC/DC converter 82A to be below the battery voltage, and subsequently adjusting the output voltage of DC/DC converter 82A to be above the battery voltage.

For some applications in which DC/DC conversion module 80 comprises exactly one DC/DC converter 82, such as described above with reference to FIG. 2A, the charging of storage capacitor 62 is performed between stimulation pulse trains 52, while current is not provided through second parallel current path 60B. For example, the one or more second-path switches 67 and second first-path switch 66 in first parallel current path 60A may be open, while first first-path switch 64 in first parallel current path 60A may be closed to connect storage capacitor 62 the DC/DC converter 82. (In configurations of the configuration shown in FIG. 2A in which two second-path switches 67 are provided, the second-path switch 67 between DC/DC converter 82 and filter capacitor 70 is typically open during charging of storage capacitor 62, to avoid charging filter capacitor 70 with the higher voltage used to by DC/DC converter 82 to charge storage capacitor 62.)

For example, storage capacitor 62 may be charged to a greater voltage than filter capacitor 70, such as least 200% of the voltage of filter capacitor 70, e.g., storage capacitor 62 may be charged to 14 V and filter capacitor 70 to 2.7 V. During discharge of storage capacitor 62, the voltage may drop to about 11 V (and/or by about 3 V). For example, when current is provided through second parallel current path 60B, DC/DC converter 82 may reduce the voltage somewhat from the voltage of the battery; e.g., if the battery has a voltage of 3 V, DC/DC converter 82 may reduce the voltage to 2.7 V.

Reference is made to FIGS. 2A-B. As described above, for some applications, each treatment session includes one or more compensation adjustment stages. For some of these applications, antenna circuit 36 further comprises a compensation circuit 68 in series with transmitting coil 38. Compensation circuit 68 is configured to provide antenna circuit 36 with an adjustable resonance frequency. Microcontroller 44 is configured to tune antenna circuit 36, via compensation circuit 68, to have a resonance frequency that does not match the fixed frequency at which power amplifier 42 drives antenna circuit 36. For example, the fixed frequency may be 6.78 MHz. (6.78 MHz+/−15 kHz is one of the ISM (Industrial, Scientific, and Medical) bands, in accordance with regulatory requirements under 47CFR § 18.301.) This deliberate frequency mismatch reduces the maximum power drawn from battery 34 during power transmission to electrostimulator implant 20 during control periods 57 and discharge periods 58, thereby preventing damage to the battery or malfunction of the ECU. Generally, depending inter operating conditions and alia manufacturing variations, the compensation circuitry causes the resonance frequency to differ by about 30 kHz from the fixed transmission frequency of 6.78 MHz, for example. For some applications, during each treatment session, the compensation is measured every pulse and, if required, compensation circuit 68 is adjusted to maintain the resonant frequency of antenna circuit 36 of ECU 22 within a tolerance of the frequency of power amplifier 42.

Optionally, this tuning is performed using techniques described in US Patent Application Publication 2023/0170138 to Oron et al., which is incorporated herein by reference. Optionally, this tuning is performed by selectively including or excluding switchable capacitors in antenna circuit 36. Typically, power amplifier 42 drives antenna circuit 36 at a fixed frequency, causing the antenna circuit to oscillate at the fixed frequency regardless of the resonant frequency of the antenna circuit.

Reference is now made to FIG. 5, which is a schematic illustration of a non-stimulation data-transmission period 100 of a pulse train 102, in accordance with an application of the present invention. For example, pulse train 102 may have the features of:
a data pulse train 202, such as described hereinabove with reference to FIG. 3, and shown by way of example in FIG. 5, or
a stimulation pulse train 52, described hereinabove with reference to FIG. 4, in which case non-stimulation data-transmission period 100 may be an entirely of or a portion of control period 57 (configuration not shown in FIG. 5).

In configurations in which pulse train 102 is a data pulse train 202, non-stimulation data-transmission period 100 may be (a) an entirety of data pulse train 202 (configuration not shown) or (b) a portion of data pulse train 202, such as shown in FIG. 5.

In configurations in which non-stimulation data-transmission period 100 is only a portion of control period 57 or a portion of data pulse train 202, control period 57 or data pulse train 202, as the case may be, also includes one or more non-data power-only portions 204 before and/or after non-stimulation data-transmission period 100, such as shown in FIG. 5 for data pulse train 202. Optionally, ECU 22 is configured to transmit an “off” period 206 to indicate the transition from non-data power-only portions 204 to non-stimulation data-transmission period 100, i.e., the start of data transmission.

For some applications, ECU 22 and/or electrostimulator implant 20 implement non-stimulation data-transmission period 100, while for other applications, other wireless energy transmitters or receivers known in the art, such as for implants, implement non-stimulation data-transmission period 100.

Since implant 20 is small and limited in resources it is preferable to use the same antenna circuitry for both wireless power transfer and data communication. Since this circuitry may be tuned to a fixed frequency (e.g., 6.78 MHz), the communication between ECU 22 and implant 20 is typically performed by modulation of the power carrier. The control and discharge periods of the RF pulse can be considered as a constant envelope RF power carrier signal.

In the application shown in FIG. 5, microcontroller 44 is configured to encode data in a plurality of bursts 110 during non-stimulation data-transmission period 100 by:
configuring each of bursts 110 to include an “on” period 112, during which power is transmitted at a frequency and an amplitude, and an “off” period 114, during which power is not transmitted, such that a burst duration D_{b} of each of bursts 110 equals a sum of an “on” duration Dₒₙ of “on” period 112 and an “off” duration D_{off} of “off” period 114 of burst 110, and
transmitting single bits of the data in respective bursts, by representing each “0” value of the bits by a first burst duration D_{b1}, and each “1” value of the bits by a second burst duration D_{b2}, the second burst duration D_{b2} different from the first burst duration D_{b1}.

Implant circuitry 30 of electrostimulator implant 20 is configured to decode the data encoded in the plurality of bursts by microcontroller 44 of ECU 22.

The duration of each Doff of “off” period 114 is short enough that relatively small amount of energy stored in implant 20 is sufficient to keep the implant circuitry functioning between “on” periods 112. For example, the “off” duration Doff of each of bursts 110 is typically no more than 50 microseconds, such as no more than 40 microseconds.

In contrast to typical on-off keying (OOK), the “off” periods (power drops) themselves cannot be interpreted as a “zero” bit, since implant 20 cannot function (i.e., remain awake when not deliberately in “sleep” mode) for an extended duration of time without power. Thus, if conventional OOK were used, a long sequence of “zero” bits would cause the implant to lose power and would result in the need to restart the implant and again perform implant initialization. Thus, the time difference between the power drops is used for signaling the bits, so that a shorter time difference indicates one type of bit (e.g., a “zero” bit) and longer time difference indicates the other type of bit (e.g., a “one” bit), or vice versa. For both bit types, the time duration between power drops is longer than the drop duration, thus allowing the implant to replenish its energy before the next drop. Moreover, following the transmission of the bits during non-stimulation data-transmission period 100, the power may continue to be transmitted during a post-data non-data power-only portion 204 for enough time to bring the implant power back to its expected value before greater power is optionally transmitted during a subsequent stimulation period 54.

For some applications, the “off” duration D_{off} of bursts 110 having the first burst duration D_{b1} equals the “off” duration D_{off} of bursts 110 having the second burst duration D_{b2}.

For some applications, the “on” duration Dₒₙ of bursts 110 having the first burst duration D_{b1} is different from the “on” duration Dₒₙ of bursts 110 having the second burst duration D_{b2}.

For some applications, a ratio of the “off” duration D_{off} to the burst duration D_{b} of bursts 110 having the first burst duration D_{b1} is less than 0.5, such as less than 0.4, e.g., less than 0.3.

For some applications, a ratio of the “off” duration D_{off} to the burst duration D_{b} of bursts 110 having the second burst duration D_{b2} is less than 0.4, such as less than 0.3, e.g., equal to 0.2.

For some applications, “on” period 112 precedes “off” period 114 in each of bursts 110, such as shown in FIG. 5. Alternatively, “on” period 112 follows “off” period 114 in each of bursts 110 (configuration not shown).

For some applications, microcontroller 44 is configured to set an amplitude of stimulation period 54 to be greater than an amplitude of non-stimulation data-transmission period 100.

Alternatively or additionally, for some applications, power unit 40 is configured to provide higher power to power amplifier 42 during stimulation period 54 of each of stimulation pulse trains 52 than during non-stimulation data-transmission period 100 of each of stimulation pulse trains 52.

For some applications, microcontroller 44 is configured to drive power amplifier 42 to drive antenna circuit 36 at a single fixed frequency during stimulation period 54 and “on” periods 112 of bursts 110 of non-stimulation data-transmission period 100 of each of stimulation pulse trains 52.

For some applications, electrostimulator implant 20 comprises an energy storage element 120 (labeled in FIGS. 2A-B) capable of storing sufficient energy to power operation of electrostimulator implant 20 for up to a maximum amount of time during non-stimulation data-transmission period 100 of each of stimulation pulse trains 52. The “off” duration D_{off} of each of bursts 110 is less than the maximum amount of time.

Alternatively, ECU 22 and implant 20 may configured to communicate using other non-keying techniques, such as modulation of the carrier amplitude or phase shifting, such as in configurations in which the clocks of the implant and ECU are synced.

### Dummy Stimulation Pulse Trains

Reference is again made to FIG. 3, and is additionally made to FIG. 6, which is a schematic illustration of dummy stimulation pulse trains 252, in accordance with an application of the present invention. FIGS. 3 and 6 inter alia provide schematic illustrations of two implementations of dummy stimulation pulse trains 252, in accordance with respective applications of the present invention. Electrostimulation system 10 may optionally use dummy stimulation pulse trains 252 to enable ECU 22 to transmit energy at times when the implant does not apply stimulation to electrostimulator implant 20 at the higher amplitude of stimulation periods 54, rather than, for example, the lower amplitude of non-stimulation data-transmission periods 100, e.g., control periods 57 or data pulse trains 202. The implant uses the received energy to power its operations and avoid falling to sleep, rather than to apply stimulation to the subject's tissue. Any excess energy not needed for the functioning of the implant is discharged by the implant without applying electrical stimulation to the subject's tissue.

To this end, microcontroller 44 of ECU 22 is configured to transmit power to electrostimulator implant 20 in a plurality of dummy stimulation pulse trains 252, by activating power unit 40 to provide the current to transmitting coil 38 of antenna circuit 36 to induce an induced current in receiving coil 26. Each of dummy stimulation pulse trains 252 includes a lower-amplitude period 260 followed (optionally, contiguously) by a higher-amplitude period 246. For example, lower-amplitude period 260 may be control period 57, described hereinabove with reference to FIG. 4. Lower-amplitude period 260 includes a data-transmission period, such as non-stimulation data-transmission period 100, described hereinabove with reference to FIG. 5.

Microcontroller 44 is configured to encode data in the data-transmission period of lower-amplitude period 260 (e.g., control period 57) of each of dummy stimulation pulse trains 252 indicative of whether higher-amplitude period 246 is a stimulation period 254 (e.g., stimulation period 54, described hereinabove) or a non-stimulation period 248.

Implant circuitry 30 is configured to, during each of the dummy stimulation pulse trains 252:
receive and decode the encoded data,
in response to the decoded data indicating that higher-amplitude period 246 is stimulation period 254, drive the one or more electrodes 28 to apply electrical stimulation to tissue of the subject using the energy received during higher-amplitude period 246, and
in response to the decoded data indicating that higher-amplitude period 246 is non-stimulation period 248, not drive the one or more electrodes 28 to apply electrical stimulation to tissue of the subject.

Typically, implant circuitry 30 receives more energy during each non-stimulation period 248 than necessary for powering its functions. Therefore, implant circuitry 30 is typically configured to discharge at least a portion of the energy received during higher-amplitude period 246 (without applying electrical stimulation to the tissue of the subject). For example, implant circuitry 30 may be configured to discharge the excess energy by shorting two electrodes 28 of opposite polarity.

Microcontroller 44 of ECU 22 may be configured to set the amplitude of non-stimulation periods 248 of dummy stimulation pulse trains 252 to be either the same as the amplitude of stimulation periods 254 of stimulation pulse trains 52, or to be lower than the amplitude of stimulation periods 254 of stimulation pulse trains 52. The implant typically discharges less energy in the latter configuration, although this latter configuration may be more complex to implement (e.g., in software of ECU 22).

Optionally, microcontroller 44 is configured to encode the data transmitted during non-stimulation data-transmission periods 100 using the “on”/“off” keying described hereinabove with reference to FIG. 5.

Reference is again made to FIG. 3. For some applications, electrostimulation system 10 is configured to use dummy stimulation pulse trains 252 to provide power to electrostimulator implant 20 during the ECU positioning stage described hereinabove, in which the position of ECU 22 outside the subject's body is adjusted with respect to electrostimulator implant 20. During this positioning stage, ECU 22 must receive feedback signals from electrostimulator implant 20 to aid with the positioning, but electrostimulator implant 20 should not apply stimulation to the tissue. Electrostimulator implant 20 needs power to transmit the feedback signals, and optionally to perform other startup functions, such as internal tests to confirm implant functionality.

Therefore, for some applications, during the ECU positioning period for positioning transmitting coil 38 of ECU 22 with to respect receiving coil 26 of electrostimulator implant 20:
microcontroller 44 of ECU 22 is configured to encode the data indicative that higher-amplitude period 246 is non-stimulation period 248, and
implant circuitry 30 is configured, in response to the decoded data indicating that higher-amplitude period 246 is non-stimulation period 248, provide data, using at least a portion of the received energy, to microcontroller 44 of ECU 22 indicative of a position of transmitting coil 38 of ECU 22 with respect to receiving coil 26 of the implant.

Reference is made to FIG. 6. For some applications, electrostimulation system 10 is configured to use dummy stimulation pulse trains 252 to provide power to electrostimulator implant 20 when intervals between stimulation periods 54 of are too long for the implant to remain awake without additional power. For example, if electrostimulation system 10 is configured (e.g., using clinician programmer 24) to provide stimulation at 2 Hz, and electrostimulator implant 20 is not capable of remaining awake for 0.5 seconds without receiving power, ECU 22 may provide one or more dummy stimulation pulse trains 252 in each of the 0.5-second gaps. Thus, dummy stimulation pulse trains 252 serve as padding at low stimulation frequencies. For example, the frequency of the stimulation pulses, including both the dummy and non-dummy pulses, may be at least 8 Hz, e.g., at least 10 Hz.

Therefore, for some applications, microcontroller 44 of ECU 22 is configured to configure a first portion 270 of dummy stimulation pulse trains 252 as stimulation pulse trains 52, and a second portion 272 of dummy stimulation pulse trains 252 as non-stimulations pulse trains 274 interspersed among stimulation pulse trains 52.

### Timing and Synchronization

Typically, implant operation is timed with ECU 22 wireless power transfer. In particular, implant 20 should inject the neurostimulation current pulse during stimulation period 54 of the RF pulse. Similarly, ECU 22 should wait for the implant feedback during certain time slot of discharge period 58. Other actions are should also occur with proper timing. Thus, the timing of ECU 22 and the implant action should occur at fixed times (with margins) in both devices.

Typically, both implant 20 and ECU 22 comprise internal clocks that time their respective actions. However, these clocks are not synchronized, and their accuracy may not always be sufficient to ensure that drift does not occur in a relatively short time. In addition, the clock frequency may be inaccurate and deviate from one implant to the other, resulting in a different timing for each implant. Therefore, in some applications, a certain level of synchronization is provided.

For some applications, to provide synchronization between implant 20 and ECU 22, a “zero” time is set from which the respective clocks of implant 20 and ECU 22 start to count. This may be done, for example, using the data transmission from ECU 22 to the implant described hereinabove with reference to FIG. 5. Both ECU 22 and implant 20 are configured to identify certain “on”/“off” features, for example the end of the transmission, and time their actions based on the time elapsed from this “zero” time. Since the “on”/“off” transmission occurs every pulse, time consistency needs to be kept only during the pulse duration.

For some applications, in order to provide even better synchronization, accurate measurement of the elapsed time from “zero” is required, which requires stable and known frequency clocks in both ECU 22 and implant 20. One technique for providing such stabilization is to lock the clock frequency of ECU 22 to the RF carrier frequency and use the RF carrier frequency at the implant side as a source for timing thus creating fully synchronous timing between the implant and ECU 22. Another alternative is to use the asynchronous transmission from the implant to ECU 22 to evaluate the implant clock frequency. To do so, ECU 22 calculates the duration between the prefix and suffix of feedback received from the implant for implant feedback containing a larger number of bits. Since the number of bits in the feedback is known, ECU 22 can compare this measured value to the value expected for a nominal implant clock frequency. Using the ratio between the numbers, ECU 22 can estimate the implant clock frequency and adjust its actions so that they will be partially synchronized with the implant actions. In a sense, ECU 22 adjust its “abstract” clock frequency to fit that of the implant clock. Note that the term “abstract” is used here, because the physical ECU clock frequency stays the same, but the timing of ECU 22 actions based on the physical clock are adjusted to represent a fixed timing on an “abstract” clock.

In an embodiment, techniques and apparatus described herein are combined with techniques and apparatus described in one or more of the following applications, which are assigned to the assignee of the present application and incorporated herein by reference:
U.S. Pat. No. 8,755,893
U.S. Pat. No. 8,788,045
U.S. Pat. No. 9,186,504
U.S. Pat. No. 9,457,186
U.S. Pat. No. 9,597,521
U.S. Pat. No. 9,713,707
U.S. Pat. No. 9,764,146
U.S. Pat. No. 9,782,589
U.S. Pat. No. 9,861,812
U.S. Pat. No. 10,004,896
U.S. Pat. No. 10,105,540
U.S. Pat. No. 10,124,178
U.S. Pat. No. 10,653,888
U.S. Pat. No. 11,213,685
US Patent Application Publication 2018/0185631
US Patent Application Publication 2023/0122706
US Patent Application Publication 2023/0170138
PCT Publication WO 2011/154937
US Provisional Application 63/434, 699, filed Dec. 22, 2022

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An external control unit (ECU) for use with an electrostimulator implant that comprises a receiving coil and is implantable in a subject, the ECU comprising:
(i) a housing;
(ii) a battery coupled to the housing;
(iii) an antenna circuit, comprising a transmitting coil;
(iv) a power unit, comprising:
(a) a power amplifier, which is arranged to provide current to the antenna circuit;
(b) first and second parallel current paths, which are arranged to convey power from the battery to the power amplifier; and
(c) a storage capacitor, connected to the first parallel current path and not to the second parallel current path; and
(v) a microcontroller, which is configured to:
transmit power to the electrostimulator implant in a plurality of stimulation pulse trains, by activating the power unit to provide the current to the transmitting coil of the antenna circuit to induce an induced current in the receiving coil, wherein each of the stimulation pulse trains includes a stimulation period and one or more non-stimulation periods,
drive the power unit to charge the storage capacitor with the power from the battery at least at one or more times other than during the stimulation period of each of the stimulation pulse trains,
during the one or more non-stimulation periods of each of the stimulation pulse trains, drive the power unit to provide the power from the battery to the power amplifier via the second parallel current path and not via the first parallel current path, and
during the stimulation period of each of the stimulation pulse trains, drive the power unit to provide power from the storage capacitor to the power amplifier via the first parallel current path and not via the second parallel current path.

2. The ECU according to claim 1, wherein the microcontroller is configured to drive the power unit to charge the storage capacitor with the power from the battery only at one or more times other than during the stimulation period of each of the stimulation pulse trains.

3. The ECU according to claim 2, wherein the microcontroller is configured to set an amplitude of the stimulation period to be greater than each of one or more respective amplitudes of the one or more non-stimulation periods.

4. The ECU according to claim 2, wherein the ECU is configured to transmit more energy during the stimulation period than during each of the one or more non-stimulation periods.

5. The ECU according to claim 2, wherein the one or more non-stimulation periods include a control period before the stimulation period.

6-7. (canceled)

8. The ECU according to claim 2, wherein the one or more non-stimulation periods include a discharge period after the stimulation period.

9. The ECU according to claim 2, wherein the storage capacitor has a capacitance of at least 1 millifarad.

10. The ECU according to claim 2, wherein the power amplifier comprises a Class E power amplifier.

11. The ECU according to claim 2, wherein the power unit is configured to provide higher power to the power amplifier during the stimulation period of each of the stimulation pulse trains than during the one or more non-stimulation periods of each of the stimulation pulse trains.

12. The ECU according to claim 11, wherein the power unit is configured to provide, from the storage capacitor to the power amplifier during the stimulation period of each of the stimulation pulse trains, more power than the battery is capable of supplying.

13. The ECU according to claim 2, wherein the power amplifier is configured to drive the antenna circuit at a single fixed frequency during the stimulation period and the non-stimulation periods of each of the stimulation pulse trains.

14. The ECU according to claim 13,
wherein the antenna circuit further comprises a compensation circuit in series with the transmitting coil, and wherein the compensation circuit is configured to provide the antenna circuit with an adjustable resonance frequency, and
wherein the microcontroller is configured to tune the antenna circuit, via the compensation circuit, to have a resonance frequency that does not match the fixed frequency at which the power amplifier drives the antenna circuit.

15. (canceled)

16. The ECU according to claim 2,
wherein the first parallel current path comprises (a) a first first-path switch arranged between the battery and the storage capacitor, and (b) a second first-path switch arranged between the storage capacitor and the power amplifier, and
wherein the microcontroller is configured to:
drive the power unit to charge the storage capacitor with the power from the battery by closing the first first-path switch and opening the second first-path switch, and
drive the power unit to provide power from the storage capacitor to the power amplifier via the first parallel current path by opening the first first-path switch and closing the second first-path switch.

17. The ECU according to claim 2, wherein the power unit further comprises a filter capacitor, which is arranged in the second parallel current path and not the first parallel current path.

18. (canceled)

19. The ECU according to claim 17, wherein a capacitance of the storage capacitor is greater than a capacitance of the filter capacitor.

20. The ECU according to claim 19, wherein a ratio of the capacitance of the storage capacitor to the capacitance of the filter capacitor is at least 1,000.

21. The ECU according to claim 2, wherein the power unit further comprises a DC/DC conversion module, which (a) comprises one or two DC/DC converters, and (b) is arranged to receive the power from the battery and provide the power to the first and the second parallel current paths.

22. The ECU according to claim 21, wherein the DC/DC conversion module comprises exactly one DC/DC converter that is connected to both the first and the second parallel current paths.

23. The ECU according to claim 22, wherein the microcontroller is configured to drive the power unit to charge the storage capacitor with the power from the battery via the DC/DC conversion module, at one or more times other than during the stimulation and non-stimulation periods of each of the stimulation pulse trains.

24. The ECU according to claim 22, wherein the microcontroller is configured to set the DC/DC converter at a first output voltage when providing the power to the first parallel current path, and at a second output voltage when providing the power to the second parallel current path, the first output voltage higher than the second output voltage.

25. The ECU according to claim 21, wherein the DC/DC conversion module comprises:
a first DC/DC converter that is connected to the battery and the first parallel current path; and
a second DC/DC converter that is connected to the battery and the second parallel current path.

26. The ECU according to claim 25, wherein the first and the second DC/DC converters have first and second output voltages, respectively, the first output voltage higher than the second output voltage.

27. (canceled)

28. The ECU according to claim 25, wherein the microcontroller is configured to drive the power unit to charge the storage capacitor with power from the battery via the first DC/DC converter at the one or more times other than during the stimulation period of each of the stimulation pulse trains, the one or more times including during at least one of the one or more non-stimulation periods of each of the stimulation pulse trains.

29. An electrostimulation system comprising the ECU according to claim 2, the electrostimulation system further comprising the electrostimulator implant.

30-47. (canceled)
